(19) United States
(12) Patent Application Publication
(10) Pub. No.: US 2008/0004695 A1
(43) Pub. Date: Jan. 3, 2008

(54) EVEROLIMUS/PIMECROLIMUS-ELUTING IMPLANTABLE MEDICAL DEVICES

(75) Inventors: Gordon Stewart, San Francisco, CA (US); Gina Zhang, Temecula, CA (US); Gene Park, Santa Rosa, CA (US); Paul Consigny, San Jose, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 11/768,749

(22) Filed: Jun. 26, 2007

Related U.S. Application Data
(60) Provisional application No. 60/817,506, filed on Jun. 28, 2006.

(51) Int. Cl. A61F 2/82

(52) U.S. Cl. 623/1.46; 623/1.42; 424/425

(57) ABSTRACT

An implantable medical device including a polymer matrix containing everolimus and pimecrolimus, and methods of using the device for the treatment of vascular disease are disclosed.

### RELATED APPLICATION

This application claims benefit of and incorporates by reference U.S. Provisional Patent Application No. 60/817,506 which was filed on Jun. 28, 2006.

### FIELD OF THE INVENTION

The present invention is directed to implantable medical devices that include a polymer matrix containing everolimus and pimecrolimus.

### BACKGROUND OF THE INVENTION

The traditional method of administering therapeutic agents to treat diseases of the internal organs and vasculature has been by systemic delivery. Systemic delivery involves administering a therapeutic agent at a discrete location followed by the agent migrating throughout the patient's body including, of course, to the afflicted organ or area of the vasculature. But to achieve a therapeutic amount of the agent at the afflicted site, an initial dose substantially greater than the therapeutic amount must be administered to account for the dilution the agent undergoes as it travels through the body. Systemic delivery introduces the therapeutic agent in two ways: into the digestive tract (enteral administration) or into the vascular system (parenteral administration), either directly, such as injection into a vein or an artery, or indirectly, such as injection into a muscle or into the bone marrow. Absorption, distribution, metabolism, excretion and toxicity, the ADMET factors, strongly influence delivery by each of these routes. For enteric administration, factors such as a compound's solubility, its stability in the acidic environs of the stomach and its ability to permeate the intestinal wall all affect drug absorption and therefore its bioavailability. For parenteral delivery, factors such as enzymatic degradation, lipophilic/hydrophilic partitioning coefficient, lifetime in circulation, protein binding, etc. will affect the agent's bioavailability.

At the other end of the spectrum is local delivery, which comprises administering the therapeutic agent directly to the afflicted site. With localized delivery, the ADMET factors tend to be less important than with systemic administration because administration is essentially directly to the treatment site. Thus, the initial dose can be at or very close to the therapeutic amount. With time, some of the locally delivered therapeutic agent may diffuse over a wider region, but that is not the intent of localized delivery, and the diffused portion's concentration will ordinarily be sub-therapeutic, i.e., too low to have a therapeutic effect. Nevertheless, localized delivery of therapeutic agents is currently considered a state-of-the-art approach to the treatment of many diseases such as cancer and atherosclerosis.

Localized delivery of therapeutic agents includes using implantable medical devices, e.g., stents. Stents play an important role in a variety of medical procedures such as, for example, percutaneous transluminal coronary angioplasty (PTCA). Stents act as a mechanical intervention to physically hold open and, if desired, expand a passageway within a subject. Problems with the use of stents, however, include thrombosis and restenosis that may present several months after a particular procedure and create a need for additional angioplasty or a surgical by-pass operation.

One of the goals of a drug-eluting stent, therefore, is to prevent or reduce the process of restenosis, which occurs after stenting of a vessel. A number of cellular mechanisms have been proposed that lead to restenosis of a vessel. Two of these include the migration and proliferation of smooth muscle cells to the site of injury and the acute and chronic inflammatory response to injury and the presence of a foreign body. Most success, thus far, has been achieved with anti-proliferative drugs coated onto to stents, however, anti-proliferative drugs are unable to completely block restenosis in all vessels.

Accordingly, there is a need for more efficacious drug-eluting stent systems.

### SUMMARY OF THE INVENTION

The present invention relates to an implantable medical device that includes a polymeric matrix disposed over the device, wherein the polymeric matrix contains everolimus and pimecrolimus.

In various aspects, the polymeric matrix includes ethylene vinyl alcohol, poly(butyl methacrylate) (PBMA) or poly(vinylidene fluoride-co-hexafluoropropene) (PVDF-HFP).

Another aspect of the invention relates to a method for treating or preventing vascular disease by implanting a medical device of the invention in a vessel of a patient in need thereof. In various aspects, the vascular disease to be treated is atherosclerosis, restenosis, vulnerable plaque or a peripheral arterial disease.

Another aspect of the invention relates to a method for coating an implantable medical device using a composition that includes a polymer, everolimus and pimecrolimus to the implantable medical device and forming a coating that includes the composition on the implantable medical device.

In various aspects, the polymer includes ethylene vinyl alcohol, PBMA or PVDF-HFP.

In various aspects, the implantable medical device is a stent.

### BRIEF DESCRIPTION OF THE DRAWINGS

FIG. 1 is a graph showing the in vivo percent release of everolimus and pimecrolimus over time.

### DETAILED DESCRIPTION

The present invention provides implantable medical devices coated with a combination of an antiproliferative drug, everolimus, and an anti-inflammatory drug, pimecrolimus, and methods of using the devices for the prevention or treatment of a vascular disease.

As used herein, “implantable medical device” refers to any type of appliance that is totally or partly introduced, surgically or medically, into a patient's body or by medical intervention into a natural orifice. The duration of implantation may be essentially permanent, i.e., intended to remain in place for the lifespan of the patient; until the device biodegrades; or until it is physically removed. Presently preferred implantable medical devices include, without limitation, catheters and stents. Stents can be self-expandable stents or balloon-expandable stents. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or a combination thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers can also be used with the embodiments of the present invention, and are known to those skilled in the art.

As used herein, a material that is described as a layer “disposed over” an indicated substrate, e.g., a stent or another layer, refers to a relatively thin coating of the material applied directly to essentially the entire exposed surface of the indicated substrate. The term “disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “polymer” refers to a molecule(s) composed of a plurality of repeating structural units connected by covalent chemical bonds.

The polymeric matrix which is disposed over an implantable medical device can be a biocompatible polymer that can be biostable or biodegradable and can be hydrophobic or hydrophilic.

As used herein, “biocompatible” refers to the property of a polymer that both in its intact, as synthesized state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

Polymers that may be used to prepare matrices of this invention include, but are not limited to, poly(N-acetylglucosamine) (Chitin), Chitosan, poly(3-hydroxyvalerate), poly(D,L-lactide-co-glycolide), poly(1-lactide-co-glycolide) poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrin glue, fibrinogen, cellulose, starch, collagen and hyaluronic acid, elastin and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), poly(vinylidene fluoride), poly(vinylidene fluoride-co-hexafluoropropylene), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes and lipids.

Presently preferred polymers include ethylene vinyl alcohol, PBMA and PVDF-HFP.

The present invention provides implantable medical devices that are coated with an anti-proliferative drug, everolimus, and an anti-inflammatory drug, pimecrolimus, and methods of using the device for the treatment or prevention of vascular disease.

Everolimus is a derivative of rapamycin with immunosuppresent and anti-proliferative properties. Everolimus acts by first binding to FK506 binding protein (FKBP12). The everolimus FKBP12 complex then binds to mammalian target of rapamycin (mTOR) and blocks its activity. By blocking mTOR activity, cells are unable to pass through G₁ of the cell cycle, thereby inhibiting proliferation. mTOR inhibition also inhibits vascular smooth muscle cell migration.

Pimecrolimus is a non-steroidal drug with strong anti-inflammatory properties. Pimecrolimus acts by binding to macrophilin-12 and inhibiting calcineurin. Calcineurin is a calcium-calmodulin dependent phosphatase that is activated upon cell stimulation. Activated calcineurin dephosphorylates nuclear factors of activated T-cells (NFATs) that are found in T-cells, monocyte macrophages, endothelial cells and vascular smooth muscle cells. The activated NFATs assemble with other transcription factors, including activator protein-1 (AP-1) and the transcription factor GATA-6, then bind to the promoter regions of genes that are activated by the stimulus. Genes that are activated by calcineurin, and thereby inhibited by pimecrolimus, include those in Table I.

TABLE I

| Cell Type | Gene |
|---|---|
| Vascular smooth muscle cell | Monocyte chemo-attractant |
| | protein-1 (MCP-1) |
| T-cells | Interferon gamma, IL-2, |
| | TNF-alpha, TNF-beta |
| T-cells | Interleukin-2 |
| Endothelial cells | Tissue factor |
| Vascular smooth muscle | IL-6 |
| cells | |
| Vascular smooth muscle | Smooth muscle myosin |
| cells | heavy chain |

The present invention takes advantage of the antiproliferative properties of everolimus and the anti-inflammatory properties of pimecrolimus by providing medical devices coated with these drugs to prevent and/or treat vascular disease, e.g., for the prevention of vulnerable plaque (VP) rupture by using the everolimus-pimecrolimus combination in a coating on an implantable medical device.

When the prevention of VP is desired, the drugs are delivered to the lesion via a traditional stent or a stent that is specially designed not to rupture the fibrous cap. Suitable stents, and stent materials, are described above. The drug combination will not only reduce inflammation but will also reduce cell proliferation and migration, thereby putting less stress on the lesion and reducing the likelihood of lesion rupture and in-stent restenosis.

Stents of the invention, having the ability to elute and/or deliver both an anti-inflammatory and an anti-proliferative drug, have particularly beneficial applications for patients with diabetes, since patients with type-2 diabetes experience higher rates of restenosis than the general population. This effect is due, at least in part, to the presence of a large number of inflammatory cells, i.e., macrophages and lymphocytes, at the site of a diabetic lesion which acts to amplify restenosis.

Another aspect of the invention relates to a method for treating or preventing vascular disease. The method involves implanting a medical device according to the invention in a vessel of a patient in need thereof. In various aspects, the vascular disease to be treated is atherosclerosis, restenosis, vulnerable plaque or a peripheral arterial disease.

As used herein, “patient” refers to any organism that can benefit from the administration of a drug, i.e., everolimus and pimecrolimus. In particular, patient refers to a mammal such as a cat, dog, horse, cow, pig, sheep, rabbit, goat or a human being.

As used herein, “treating” refers to the administration of a therapeutically effective amount of a drug to a patient known or suspected to be suffering from a vascular disease. Presently preferred drugs useful with this invention include, but are not limited to, everolimus and pimecrolimus.

As used herein, “therapeutically effective amount” refers to the amount of drug that has a beneficial effect, which may be curative or palliative, on the health and well-being of a patient with regard to a vascular disease with which the patient is known or suspected to be afflicted. A therapeutically effective amount may be administered as a single bolus, as intermittent bolus charges, as short, medium or long term sustained release formulations or as any combination of these.

As used herein, “known” to be afflicted with a vascular disease refers first to a condition that is relatively readily observable and or diagnosable. An example, without limitation, of such a disease is atherosclerosis, which is a discrete narrowing of a patient's arteries. Restenosis, on the other hand, while in its latter stages, like atherosclerosis, is relatively readily diagnosable or directly observable, may not be so in its nascent stage. Thus, a patient may be “suspected” of being afflicted or of being susceptible to affliction with restenosis at some time subsequent to a surgical procedure to treat an atherosclerotic lesion.

As used herein, an “atherosclerotic lesion” refers to a deposit of fatty substances, cholesterol, cellular waste products, calcium and/or fibrin on the inner lining or intima of an artery.

As used herein, “restenosis” refers to the re-narrowing or blockage of an artery at or near the site where angioplasty or another surgical or interventional procedure was previously performed to remove a stenosis.

As used herein, “peripheral arterial disease” refers to a condition similar to coronary artery disease and carotid artery disease in which fatty deposits build up in the inner linings of the artery walls thereby restricting blood circulation, mainly in arteries leading to the kidneys, stomach, arms, legs and feet.

Vulnerable plaque on the other hand is quite different from either atherosclerosis or restenosis. Vulnerable plaque occurs primarily within the wall of a vessel and does not cause prominent protrusions into the lumen of the vessel. It is often not until it is “too late,” i.e., until after a vulnerable plaque has broken and released its components into the vessel, that its presence is even known. Numerous methods have and are being investigated for the early diagnosis of vulnerable plaque but to date none have proven completely successful.

### EXAMPLES

The following examples are provided to further teach the concepts and embodiments of the present invention. They are not intended nor are they to be construed in any manner to limit the scope of the present invention.

### Example 1

### EVAL-based Everolimus and Pimecrolimus Coated Stent

A 12 mm Vision™ stent was cleaned in a bath of isopropyl alcohol and treated in a plasma cleaner to strip the top layer of molecules from the stent for better coat adhesion. The stent was then spray-coated with a primer solution consisting of 4% Poly(ethylene-co-vinyl alcohol) (EVAL) in a 80:20 N,N-dimethylacetamide (DMAC):pentane solution. The coated stent was dried in a convection oven at 140° C. for 1 hour.

The stent was then spray-coated with a drug reservoir solution consisting of EVAL, pimecrolimus and everolimus. A drug:polymer ratio of 1:2 of both pimecrolimus and everolimus to EVAL was used to coat the stent in order to obtain 64 μg of pimecrolimus and 64 μg of everolimus in the final coating. Spray coating parameters and the solvent were identical to those used for coating with the primer layer.

The total content and percent recovery of everolimus and pimecrolimus on the dried stent was measured using methods known in the art. In the present example, the theoretical total content of everolimus on the stent was 62.5 μg while the actual total content was 60.6 μg, equaling a 97.0% recovery of everolimus. The theoretical total content of pimecrolimus was 62.5 μg while the actual total content was 59.1 μg, equaling a 94.6% recovery of pimecrolimus.

It was also observed under scanning electron microscopy that no flaking or delimination of the dual-coated stents occurred confirming that the stent would be acceptable for patient implantation.

### Example 2

### Everolimus and Pimecrolimus Release From Dual-Coated Stent

Using the method set forth in Example 1, a 12 mm Vision™ stent was similarly coated with everolimus and pimecrolimus. The 24 hour release of everolimus and pimecrolimus in porcine serum was then measured using methods known in the art. It was found that 12.2 μg of an expected 62.3 μg of everolimus was released over 24 hours which resulted in 19.7% release. For pimecrolimus, 12.4 μg of an expected 60.6 μg of pimecrolimus was released over 24 hours resulting in 20.5% release.

### Example 3

### PVDF-HFP-based Everolimus and Pimecrolimus in vivo Release Rate

A 12 mm Vision™ stent was cleaned in a bath of isopropyl alcohol and treated in a plasma cleaner to strip the top layer of molecules from the stent for better coat adhesion. The stent was then spray-coated with a primer solution consisting of 2% PBMA. The coated stent was dried in a convection oven at 80° C. for 0.5 hours.

The stent was then spray-coated with a drug reservoir solution consisting of PVDF-HFP, pimecrolimus and everolimus. A drug:polymer ratio of 1:3.5 of both pimecrolimus and everolimus to PVDF-HFP was used to coat the stent in order to obtain 64 μg of pimecrolimus and 64 μg of everolimus in the final coating. Spray coating parameters and the solvent were identical to those used for coating with the primer layer.

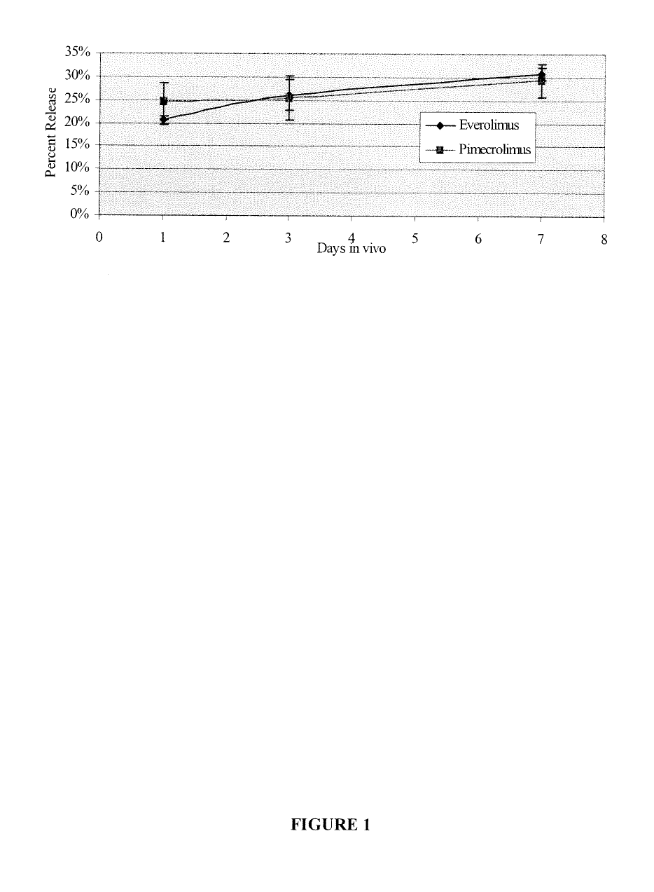

The stent was implanted in a pig and 1, 3 and 7 day release rates were obtained and compared with 24 hour release rates in porcine serum. FIG. 1 shows the in vivo percent release of everolimus and pimecrolimus over time. By day 7, release of both drugs was 30%.

In summary, the present invention provides for the simultaneous incorporation and elution of everolimus and pimecrolimus from polymers, such as EVAL and PVDF-HFP, with a suitable recovery of both drugs. The everolimus/pimecrolimus coatings have acceptable coating integrity and their elution rates can be controlled. Specifically, it was found that formulations could simultaneously release both drugs from rates of about 10% to about 40% in 24 hours in porcine serum (“PS”). In addition, it was seen that everolimus and pimecrolimus can be made to elute simultaneously in vivo from a PVDF-HFP polymer matrix, with a near 1:1 correlation with PS release.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention.

1. An implantable medical device comprising:
a polymeric matrix disposed over the device, wherein the polymeric matrix comprises everolimus and pimecrolimus.

2. The implantable medical device according to claim 1, wherein the polymeric matrix comprises ethylene vinyl alcohol, poly(butyl methacrylate) or poly(vinylidene fluoride-co-hexafluoropropene).

3. The implantable medical device according to claim 1, wherein the implantable medical device is a stent.

4. A method for treating or preventing vascular disease comprising:
implanting the medical device according to claim 1 in a vessel of a patient in need thereof.

5. The method according to claim 4, wherein the vascular disease is atherosclerosis, restenosis, vulnerable plaque or a peripheral arterial disease.

6. A method for coating an implantable medical device comprising:
applying a composition comprising a polymer, everolimus and pimecrolimus to the implantable medical device; and
forming a coating comprising the composition on the implantable medical device.

7. The method according to claim 6, wherein the polymer comprises ethylene vinyl alcohol, poly(butyl methacrylate) or poly(vinylidene fluoride-co-hexafluoropropene).

8. The method according to claim 6, wherein the implantable medical device is a stent.
